(19) United States
(12) Patent Application Publication
(10) Pub. No.: US 2018/0161198 A1
(43) Pub. Date: Jun. 14, 2018

(54) DEVICE FOR HEATING A PATIENT BEARING AREA OF AN OPERATING TABLE

(71) Applicant: MAQUET GMBH, RASTATT (DE)

(72) Inventors: Immanuel Gaiser, Durmersheim (DE); Bernhard Katzenstein, Iffezheim (DE)

(73) Assignee: MAQUET GMBH, RASTATT (DE)

(21) Appl. No.: 15/883,013

(22) Filed: Jan. 29, 2018

Related U.S. Application Data
(63) Continuation-in-part of application No. PCT/EP2016/067397, filed on Jul. 21, 2016.

(30) Foreign Application Priority Data
Jul. 30, 2015 (DE) 10 2015 112 449.1

(51) Int. Cl. A61F 7/00; A61B 6/04

(52) U.S. Cl. CPC A61F 7/0053; A61F 2007/0054; A61F 2007/0055; A61B 6/045

(57) ABSTRACT

Devices, arrangements, and methods for heating a patient bearing area of an operating table, including a heating element for heating a patient bearing area of an operating table, with a heating element for heating a fluid that transfers heat, an elastically deformable flow layer of the patient bearing area through which the heated fluid can flow, and a fluid-tight sealing layer of the patient bearing area arranged between a surface of the patient bearing area provided for contact with the patient and said flow layer. The device may include a flow circuit for the circulating, heat-bearing fluid formed as a closed path.

### CROSS REFERENCE TO RELATED APPLICATIONS

The present application is a continuation-in-part filed under 35 U.S.C. § 111(a), and claims the benefit under 35 U.S.C. § 365(c) of PCT International Application PCT/EP2016/067397, filed Jul. 21, 2016, which designates the United States of America, and claims the benefit of German Patent Application No. DE 10 2015 112 449.1, filed Jul. 30, 2015. Both applications are incorporated by reference in their entirety.

### FIELD OF THE DISCLOSURE

The present disclosure relates to devices and arrangements for heating a patient bearing area of an operating table with a heating element. It also relates to tables incorporating such heating devices and arrangements, and to methods of using such devices, arrangements, and tables.

### BACKGROUND

During operations it can be problematic to maintain a suitable body temperature for the patient. Especially during operations requiring incisions in the thorax, in the abdomen, or on the legs, the patient might not be adequately covered for thermal insulation (e.g. for reasons of protecting against infection), and hypothermia may result. Furthermore, during certain operations, such as heart surgery, an artificial hypothermia of the patient is brought about by medications which affect the patient's temperature regulation.

From U.S. Pat. No. 6,653,607 B2 there is known a heating element operated with electric current and embedded in a mattress, which conductively transfers heat to the mattress, so that heat is likewise supplied by conductive heat transfer to the patient lying on the mattress. Providing a mattress separate from the patient bearing area for warming the patient results in increased expenses, e.g. for fixation of the mattress on the patient bearing area, and fixation of the patient on the mattress. Further, both the mattress and the patient bearing area must be cleaned and disinfected. Moreover, the heating element of the mattress, conduction paths, and sensors can appear during imaging procedures performed on the patient, which complicates diagnostics. Therefore, there is a need for heating arrangements using materials of low density, which are transparent to X-rays and other imaging methods, including at low radiation intensity.

During long operations decubitus (e.g. bedsores) can occur in regions of the patient resting forcefully on the patient bearing area. Decubitus problems are worsened by heating of the decubitus-threatened regions of the patient by a mattress. Ideally, heat transfer should occur only in regions of the patient resting with slight pressing force on a patient bearing area. Such differentiated heat transfer cannot be assured by a heating element embedded in a mattress.

Protection of the patient against excessive heating is difficult due to typically uneven pressing force of the patient on the mattress when using an electrically operated heating element. Correction requires reliable monitoring of the temperature across the entire possible bearing area combined with complex compensatory adjustments to heating.

Patients must also be protected against electric currents when using direct electrical heat for beds and mattresses.

DE 20 2006 017 369 U1 describes a thermal blanket in which warm air is introduced, which air in turn emerges from the bottom side of the thermal blanket facing the patient and warms the patient. The emerging air warms the surroundings as well as the patient, so that the surgeons are also exposed to the warm air flow during an operation. Furthermore, the risk of wound infection is increased by pathogens taken up or carried along with the emerging air.

### SUMMARY OF THE DISCLOSURE

Embodiments of the disclosure include a device for heating a patient bearing area by which heat can be supplied to the patient in a simple and gentle manner Embodiments include a device for heating a patient bearing area of an operating table.

In some embodiments neither the fluid, nor the flow layer, cause distortion of the picture in an imaging procedure, so that the suitability of the patient bearing area for a radioscopy of the patient is increased. In some embodiments, the fluid and the flow layer are radio transparent and do not appear in patient imaging procedures. In some embodiments, regions of heated patient bearing areas which are under or otherwise aligned with areas of a patient to be imaged are formed primarily or exclusively of radio transparent materials and/or materials which will not appear or substantially will not appear during patient imaging procedures. Embodiments include arrangements and methods of simultaneously heating and imaging a patient, where the heating arrangement does not appear in or interfere with captured images of the relevant region of the patient. For example, heat-supplemented methods and non-image-interfering heating arrangements for: Computed Tomography (CT), also referred to as a CAT scan, and/or Magnetic Resonance Imaging (MRI), and/or Positron Emission Tomography (PET) scans, and/or combined PET/CT scans, and/or X-rays, and/or ultrasound (also known as medical sonography or ultrasonography), or combinations of these techniques.

In some embodiments, the patient bearing area has an elastically deformable flow layer through which heated fluid can flow. By flowing of the heated fluid through this flow layer, heat can be supplied to the patient in simple and gentle manner. Overheating of individual regions of the patient can be avoided, since the temperature of the fluid flowing through the flow layer of the patient bearing area, and/or the flow rate of fluid created through the flow layer, are preferably adjustable. In some embodiments, due to elastic deformability of the flow layer, the volume flow of fluid in the regions of the flow layer more heavily deformed or compressed by the pressing force of the patient is reduced as compared to the other regions, and therefore the patient bearing area is automatically less heated in those regions. Decubitus-prone regions of the patient are therefore automatically heated less than the other regions, which protects the patient. The elasticity of such flow layers combines simple construction of the patient bearing area and a comfortable support of the patient. In some applications, a further elastic layer to ease the pressure of the patient supported between the flow layer and the surface of the patient bearing area is rendered unnecessary. Thus, in some arrangements, the elastic flow layer is the only cushioning supporting the patient, i.e. there is no additional elastic layer between the patient and the nearest underlying rigid structure. Where the flow layer is integrated into and/or provides the patient bearing area, preferably no additional bearing elements or blankets need to be handled or cleaned.

In embodiments of the invention, fluid flowing through the flow layer transfers heat to the patient bearing area, which in turn transfers heat to the patient conductively across its surface. The fluid provided for flow through the flow layer is preferably a gas, such as air, or a liquid, such as water or treated water. The flow layer may be an open-pore (aka open-cell) foam material, a fleece, an inflatable structure (e.g. a flat inflatable structure) inflatable by the heat transfer fluid, and/or a three-dimensional textile structure. Under compression pressure by the weight of a patient, the cross-section area of the flow layer may become smaller, so that the flow cross section is reduced.

In some embodiments the flow layer is be formed partially or fully by an open-pore/open-cell foam material. This ensures that the flow layer can be flowed through and be elastically deformable at the same time. Polyurethane is an available option for the open-cell/open-pore foam.

In some typical embodiments, the flow layer has at least one entry region for introducing fluid heated by the heating element into the flow layer, and the flow layer has at least one exit region for the emergence of the fluid from the flow layer. In this way, the heating element can be provided outside the flow layer. The entry region(s) and exit region(s) may be provided spaced apart on the flow layer. For example, at or near opposite edges, at the corners, or other arrangements to provide flow across a maximum area of the flow layer. For example, the exit and/or entry regions can be positioned so that they begin no more than 0.5, 1, 1.5, 2, 3, or 4 inches from the nearest edge of the bed, segment or patient bearing area. Alternatively, the exit and/or entry regions can be positioned, with respect to the nearest edge, so that they begin no more than 5%, 10%, 15%, or 20% of the distance across the bed, patient bearing region, or segment. In some embodiments the entry region(s)/feed opening(s) are at least 4, 6, 8, 10, or 12 inches away from the nearest exit region(s)/exit opening(s) in order to provide a long flow path for fluid through the flow layer for heat transfer to a patient.

In some applications, providing the heating and/or circulating elements outside the flow layer provides the advantages that they do not impair the elastic deformability of the flow layer, and/or that they can be positioned outside of an area being imaged to avoid affecting the image. For example, the flow layer may be separated from the heating and/or circulating units by a rigid internal support plate.

In advantageous embodiments, a flow generator is provided, by means of which a fluid flow can be created for the introducing of fluid into the entry region(s) of the flow layer, for the flow through the flow layer, and for the emergence from the exit region(s) of the flow layer. This provides an effective recirculation of the fluid and the most uniform possible heat transfer from the fluid to the flow layer. When air is provided as the fluid, the flow generator could be a fan or a blower. When a liquid is provided as the fluid, a pump could be provided as the flow generator.

In some embodiments the patient bearing area has at least one bearing area segment with a cushion. The surface of the bearing area segment can be a surface of the cushion of the bearing area segment. A flow layer and a sealing layer can be part of the cushion of the bearing area segment. This enables modular design of the patient bearing area, e.g. with several bearing area segments, of which the cushion of one or more bearing area segments is heated. Patient bearing areas and corresponding beds could be selectably provided with a combination of heated and non-heated cushion segments. In some embodiments, modular heated cushion segments are self-contained, each having flow layer(s) and their own respective heat and flow generators.

In some embodiments, the surface of the patient bearing area provided for contact with the patient is a surface of the sealing layer. This provides a rapid and effective heat transfer surface from the flow layer to the patient. The sealing layer may be, without limitation, a polyurethane spray skin.

A heat storage layer may be provided adjacent to the flow layer for storage of heat from the fluid. In some embodiments heat transferred from the flow layer to regions of the patient bearing area not making contact with the patient is stored in the heat storage layer, and a longer time of transfer of heat to the patient is made possible after switching off the heating element. Heat storage layers may work by latent heat storage using wax, using supersaturated solutions, and/or utilizing other materials with high caloric capacity, such as silicone gels, polyurethane gels, water mats, or oil mats. The heat storage layer may be arranged in the flow layer, below the flow layer, and/or between the patient and the flow layer. The heat storage layer may function as a heat sink buffer, slowing temperature changes while and after active heating is employed.

In some embodiments the at least one entry region (e.g. feed opening) and the at least one exit region (e.g. exit opening) are arranged outside a region of the patient bearing area intended for radioscopy or other imaging of the patient. For example, the entry and exit regions can be positioned lateral to areas of the segment which support areas of the patient to be imaged, so that they are not below or aligned with areas to be imaged. This prevents the entry region or the exit region from disturbing or appearing in pictures created during radioscopy or other imaging of the patient. The exit and/or entry regions can be positioned near lateral edges of the arrangement to reduce interference with imaging and/or to provide a flow path spanning most of the heated segment. For example, the exit and/or entry regions can be positioned so that they begin no more than 0.5, 1, 1.5, 2, 3, or 4 inches from the nearest edge of the bed, segment or patient bearing area. Alternatively, the exit and/or entry regions can be positioned, with respect to the nearest edge, so that they begin no more than 5%, 10%, 15%, or 20% of the distance across the bed, patient bearing region, or segment.

In some embodiments the heating element is arranged in the patient bearing area. For example, in a heated bearing area segment. This accomplishes a compact arrangement of the device and protects the heating element against contamination by the surface of the patient bearing area. Heat transfer from the heating element to the fluid may occur via a heat exchanger connected to the heating element.

In can be advantageous for the heating element to be an X-ray transparent surface heating element, and for the fluid when the flow generator is activated to flow past the heating surface of the surface heating element. This accomplishes an effective heat transfer to the fluid. Alternatively, or additionally, the heating element (optionally a single unit with a circulating element) can be arranged in a marginal region of the patient bearing area, especially in a marginal region of a cushion of the patient bearing area. A patient will preferably be positioned on the patient bearing area such that he is not lying in the marginal region, or at least such that no relevant regions of the patient undergoing radioscopy are positioned in the marginal region aligned with or over the heating element. For example, the heating element may be positioned in a lateral area of the patient bearing region and/or of a given heated segment adjacent an edge. For example, extending no more than 2, 3, 4, 5, or 6 inches from the nearest edge at maximum. Alternatively, not extending away from the nearest edge by more than 10%, 15%, 20%, 35%, or 30% of the distance across the bed, patient bearing region, or segment.

In some embodiments the entry region is arranged at a first end of the bearing area segment, and the exit region is arranged at a second end of the bearing area segment opposite the first end, so they are spaced apart in the longitudinal direction of the patient bearing area. Longitudinally spaced apart openings providing a longitudinal flow can be advantageous. This is because a longitudinally oriented patient can make an elastic deformation of the flow layer along the longitudinal axis of the patient bearing area. Such deformation might impede a lateral flow by forming a “wall” of compressed flow layer fully across the segment, under the patient.

In some embodiments, it is advantageous for the bearing area segment to have a second entry region for the introducing of fluid heated by the heating element into the flow layer, and for the bearing area segment to have a second exit region for the emergence of the fluid from the flow layer. In this way, a more uniform heating of the bearing area segment can be achieved.

In some embodiments a control unit and a valve system are provided for controlling the fluid flow through the flow layer. The valve system in this case can be controlled by the control unit such that the fluid is introduced only in an entry region into the flow layer. This makes possible a timed sequencing of different flow patterns through the flow layer, so that the bearing area segment can be heated in a flexible manner

It can be advantageous to provide a cleaning unit for the cleaning of contaminated fluid. This prevents an accumulation of pathogens in the fluid, which might get into the body of the patient. The cleaning unit is preferably suitable to removing the pathogens from the fluid and/or inactivating them. Preferably, the cleaning unit ionizes and/or filters the fluid.

In certain embodiments, the bearing area segment is a first bearing area segment, wherein the first bearing area segment has a first port for the introducing of the fluid into the first bearing area segment and a second port for the emergence of the fluid from the first bearing area segment. Furthermore, the patient bearing area has at least one second bearing area segment with a first port for the introducing of the fluid into the second bearing area segment and one second port for the emergence of the fluid from the second bearing area segment. The second bearing area segment also has an elastically deformable flow layer through which heated fluid can flow and a fluid-impermeable sealing layer arranged between a surface of the second bearing area segment designed for patient contact and the flow layer of the second bearing area segment. In some embodiments, the flow layer of the second bearing area segment has at least one entry region for the introducing of the fluid heated by the heating element into the flow layer of the second bearing area segment and at least one exit region for the emergence of the fluid from the flow layer of the second bearing area segment. The second port of the first bearing area segment is connected to the first port of the second bearing area segment such that the fluid, when the flow generator is activated, is introduced by the first port of the first bearing area segment into the first bearing area segment, flows from the first bearing area segment into the second bearing area segment, is introduced into the entry region of the flow layer of the second bearing area segment, emerges from the exit region of the flow layer of the second bearing area segment and emerges from the second port of the second bearing area segment. This accomplishes a guidance of the fluid from the first bearing area segment into the second bearing area segment, so that only one heating element need be provided for the heating of several bearing area segments. The guidance of the fluid may be extended accordingly to a third bearing area segment or further bearing area segments. Preferably a star-shaped construction, a ring-shaped construction, or spur lines will be used for this.

Advantageous embodiments include a closed fluid circulation, in which the flow layer, the heating element, and the flow generator are arranged. By providing such a closed system, the penetrating of pathogens into the fluid or other regions of the device is more difficult. The closed fluid circuit is fluid-tight against the outside.

In some embodiments air is provided as the fluid, and can be sucked in from the surroundings by means of the flow generator. When the flow generator is activated, air sucked in from the surroundings is introduced into the entry region of the flow layer of the bearing area segment. This will achieve an especially simple layout and an economical manufacturing of the device. The air introduced in the entry region of the flow layer emerges from the flow layer, after being heated, and emerges from the bearing area segment. The system resulting from this layout has an open circuit.

An embodiment of the device includes a bed or a bearing area segment containing a fluid flow path for liquid or gas, and a method including heating fluid while circulating it through the fluid flow path. While the specific path may vary, the flow path can preferably be embodied as at least one circuit. The flow path can be embodied as (i) a flow layer where heated fluid passes near a patient or a patient bearing surface to provide heat there to; then (ii) exit opening(s) for liquid leaving the flow layer (e.g. downwards); then (iii) a heat generating unit and recirculation unit (in either sequence, and optionally combined in a single module) heating the fluid and impelling the fluid through the circuit, respectively; then (iv) flow channels(s) transporting liquid towards (v) feed opening(s) for liquid to return (e.g. upwards) to the flow layer. The flow layer may be open cell/open-pore foam. The flow layer can be generally planar and/or generally horizontal. The flow channel(s) can be generally horizontal. Instead of a flow channel, a flow space having a shape other than a channel shape is contemplated. In a preferred embodiment the flow layer is planar, and one or more flow channels pass liquid through a different plane which is parallel to the planar flow layer. The feed opening(s) and exit opening(s) may be embodied as passages which are perpendicular to the planes of the flow layer and the flow channel(s). There may be flow channel(s) upstream and/or downstream of the heat generating unit and recirculation units

An embodiment of the device employs a fluid flow path, potentially with branches and parallel paths, which require all or substantially all of the fluid to pass laterally through a planar flow layer. For example, a flow layer in the form of a generally flat section of open cell/open pore foam shaped to provide at least part of a patient bearing area of a surgical table. Heat leaves the liquid to warm a patient bearing area while the liquid is in the flow layer. After passing laterally through at least part of the flow layer, the cooled fluid leaves the flow layer via one or more exit openings. The exit openings may facilitate movement of the liquid away from the patient-facing surface of the patient bearing area. Liquid could then proceed to flow channels/cavities, flow generator(s), and heater(s) which can potentially be in any sequence relative to each other. The flow generator, heater, and channels can be located away from the patient facing surface of the patient bearing area. The flow generator (e.g. a pump or fan) impels the liquid to maintain circulation through the flow path. The heater reheats the liquid. The channel(s) direct the liquid to one or more feed openings where warmed liquid moves back towards the flow layer in a location spaced apart from the exit openings. The liquid then flows through the flow layer again and the sequence is repeated as long as the device is turned on. In some embodiments liquid flow through the flow layer and through the flow channels/cavities is parallel but in generally opposite directions. In some embodiments liquid flow through the feed openings and exit openings is also mutually parallel but in generally opposite directions. Methods of heating a patient and/or a patient bearing area of a surgical table, and devices and tables for use in such heating, are contemplated.

One useful embodiment includes a heated operating table for holding a patient during a medical procedure, the operating table comprising: a patient bearing area for holding the patient thereon, the patient bearing area comprising a plurality of bearing area segments for collectively supporting the patient, wherein at least some of the bearing areas segments each comprise a planar top surface oriented for supporting the patient, and wherein the bearing area segments comprise a heated segment for warming the patient. An exemplary heated segment includes: a circulating fluid enclosed therein, a top surface, the top surface being fluid-tight, and being oriented generally upwards for supporting the patient; a flow layer below the top surface, the flow layer comprising open-cell foam, and being elastically deformable; a dividing member below the flow layer, the dividing layer being made of fluid impermeable material, and having a feed opening and an exit opening there through; and a flow channel below the dividing member. The device can also include a heat generating unit for heating the circulating fluid, the heat generating unit being below the dividing member; and a recirculating unit located below the dividing member, and oriented for impelling the circulating fluid through a flow circuit in a single downstream direction. In some embodiments the flow circuit is a closed path within the heated segment for the circulating fluid, the flow circuit being collectively formed by at least the following elements: the flow layer, the exit opening of the dividing member, the flow channel, and the feed opening of the dividing member. I useful embodiments the recirculating unit and the heat generating unit are each positioned either in or adjacent to the flow channel for, respectively, impelling and heating the circulating fluid when the circulating fluid is not within the flow layer. The flow circuit can be arranged so that during operation heated circulating fluid enters the flow layer and flows there through for providing heat to the top surface, and so that circulating fluid thereafter leaves the flow layer and returns to the heat generating unit. Embodiments include one or more valves positioned to prevent circulating liquid from at least one of (i) leaving the flow layer via the feed opening or (ii) entering the flow layer via the exit opening.

In some embodiments the dividing member is a rigid, planar, support plate having first and second edges at opposite ends thereof. In some embodiments the support plate comprises a feed opening there through for circulating fluid entering the flow layer, and an exit opening there through for fluid leaving the flow layer. The feed opening can be within one, two, three, or four inches of the first edge of the support plate, and/or at least part of the exit opening can within one, two, three, or four inches of the second edge of the support plate. In some embodiments the feed opening and exit openings are spaced at least three, five, eight, or twelve inches apart from each other.

In some cases the heated segment comprises a bottom plate positioned below both the flow layer and the dividing member, with the dividing member located between the flow layer and the bottom plate, and at least part of the flow channel is in the form of a concave cavity in the bottom plate. The heat generating unit and/or the recirculating unit may both be located between the bottom plate and the dividing layer. The flow channel can be a channel fluidically connecting the exit opening to the feed opening, with the recirculating unit located in the flow channel and oriented for impelling circulating fluid downstream towards the feed opening.

In some embodiments the dividing member comprises a plurality of feed openings and a plurality of exit openings, and the feed openings are each at least three, six, nine, or twelve inches away from the nearest exit opening.

In some embodiments at least the circulating fluid and the flow layer of the heated segment are X-ray transparent. The heat generating unit may comprise an X-ray transparent surface heating element positioned along the flow circuit for heating circulating fluid therein. The feed opening and the exit opening may be positioned outside an area of the heated segment which is within a patient imaging area.

In some embodiments the heat generating unit and the recirculating unit are provided in a single combined unit, the combined unit having a heating passage for flow of circulating fluid there through, the combined unit being configured to both heat and compel circulating fluid passing through the heating passage.

In some applications the heated segment is generally planar, having a maximum width at least three, four, five, six, or eight times greater than a maximum thickness.

The disclosure includes methods using the devices described throughout this disclosure. For example, a method of heating a patient in need thereof, the method comprising: providing the heated operating table of claim 1, and positioning the patient on the heated operating table; heating circulating fluid in the flow circuit of the heated segment using the heat generating unit; impelling the circulating fluid in a downstream direction with regard to the flow circuit using the recirculating unit; wherein the circulating fluid impelled by the recirculating unit proceeds through the flow channel, then through the feed opening, then through the open-cell foam of the flow layer, then out of the flow layer via the exit opening, and then returns to the recirculating unit; and wherein heat is transferred from circulating fluid passing through the flow layer, through the top surface of the heated segment, to the patient on the heated operating table.

### BRIEF DESCRIPTION OF THE DRAWINGS

FIG. 1 is a schematic perspective view of a patient bearing area;

FIG. 2 is a perspective view of a bearing area segment of a first embodiment;

FIG. 3 is a perspective view of the bearing area segment in FIG. 2, with the cushion unit and support plate shown separated for purposes of illustration, so that internal elements are visible;

FIG. 4 is a partly sectioned perspective view of the bearing area segment of FIGS. 2-3;

FIG. 5 is an enlarged representation of the region A indicated in FIG. 4;

FIG. 6 is a cross section of the bearing area segment in FIGS. 2-5 along a vertical sectioning plane transversely to the patient bearing area;

FIG. 7 is an enlarged representation of a region B of the bearing area segment in FIG. 6;

FIG. 8 is a cross section of a bearing area segment of a device for heating the bearing area segment according to a second embodiment;

FIG. 9 is an enlarged representation of a region C of the bearing area segment indicated in FIG. 8;

FIG. 10 is a perspective representation of a partly sectioned enlarged cutout of a bearing area segment according to a second embodiment;

FIG. 11 is a cross section of a bearing area segment of a device for heating a bearing area segment according to a third embodiment;

FIG. 12 is an enlarged representation of a region D of the bearing area segment indicated in FIG. 11 according to the third embodiment;

FIG. 13 is a perspective representation of a partly sectioned enlarged cutout of the bearing area segment according to the third embodiment;

FIG. 14 is a top view of a bearing area segment of a device according to a fourth embodiment;

FIG. 15 is a view of the bottom of the bearing area segment according to the fourth embodiment;

FIG. 16 is a schematic perspective representation of a cross section of the bearing area segment and an external heat generating and recirculation unit according to the fourth embodiment;

FIG. 17 is a top view of a schematically represented patient bearing area, having a bearing area segment receiving a flow transversely to the longitudinal axis of the patient bearing area;

FIG. 18 is a top view of another schematically represented patient bearing area, having a bearing area segment receiving a flow of air in the direction of the longitudinal axis of the patient bearing area;

FIG. 19 is a top view of another schematically represented patient bearing area, in a first configuration for the flow through a bearing area segment;

FIG. 20 is a top view of a patient bearing area in a second configuration for flow through a bearing area segment;

FIG. 21 is a top view of a patient bearing area in a third configuration for flow through a bearing area segment;

FIG. 22 is a schematic perspective view of a device for heating of two bearing area segments sequentially receiving a flow of a fluid according to another embodiment; and

FIG. 23 is a schematic perspective view of an operating table having a patient bearing area.

### DETAILED DESCRIPTION

For illustrative purposes, the principles of the present invention are described by referencing various exemplary embodiments. Although certain embodiments of the invention are specifically described herein, one of ordinary skill in the art will recognize that the same principles are equally applicable to, and can be employed in, other systems and methods. It should be understood that the invention is not limited in its application to the details of any particular embodiment shown. Additionally, the terminology used herein is for the purpose of description and not of limitation. In this disclosure, the singular forms “a”, “an”, and “the” include plural references unless the context clearly dictates otherwise. Similarly, the terms “a” (or “an”), “one or more” and “at least one” can be used interchangeably herein. It is also to be noted that the terms “comprising,” “including,” “composed of,” and “having” should be interpreted interchangeably in the written description. Elements and steps in particular embodiments in the description may be used with elements disclosed in other embodiments. Elements with similar or identical construction and/or function may have the same reference numbers.

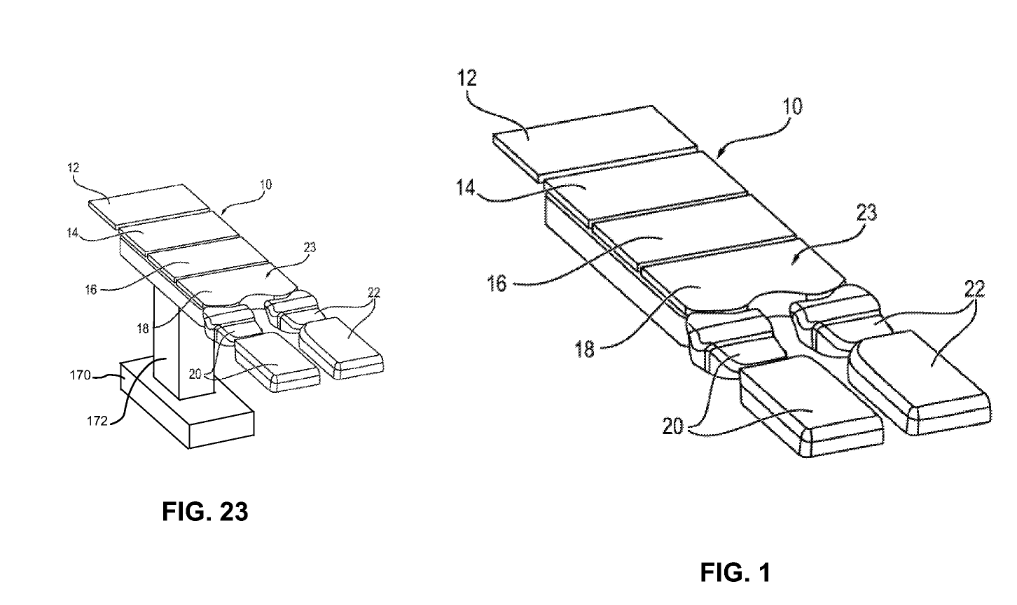

FIG. 1 shows a schematic perspective view of a patient bearing area 10 according to a first embodiment. The patient bearing area may be part of a table such as an operating table, including a base 170 and a support column 172, as shown in FIG. 23. The Patient bearing area 10 has several bearing area segments which are adjustable in their position, enabling various positioning of a patient, beyond those specifically illustrated. In the present sample embodiment, the bearing area segments of the patient bearing area 10 comprise a head plate 12, a back plate 14, a torso plate 16, a pelvis plate 18, a two-piece right leg plate 20 and a two-piece left leg plate 22, of which the pelvis plate 18 is designed for example as a bearing area segment of a device 23 for heating the patient bearing area 10 according to the invention. In other embodiments, a device 23 for the heating of the patient bearing area 10 according to the invention may comprise further bearing area segments in identical fashion, alternatively or additionally, like the pelvis plate 18. Heated bearing area segments can be deployed in various shapes, positions, and arrangements, typically in combination with non-heated segments or areas.

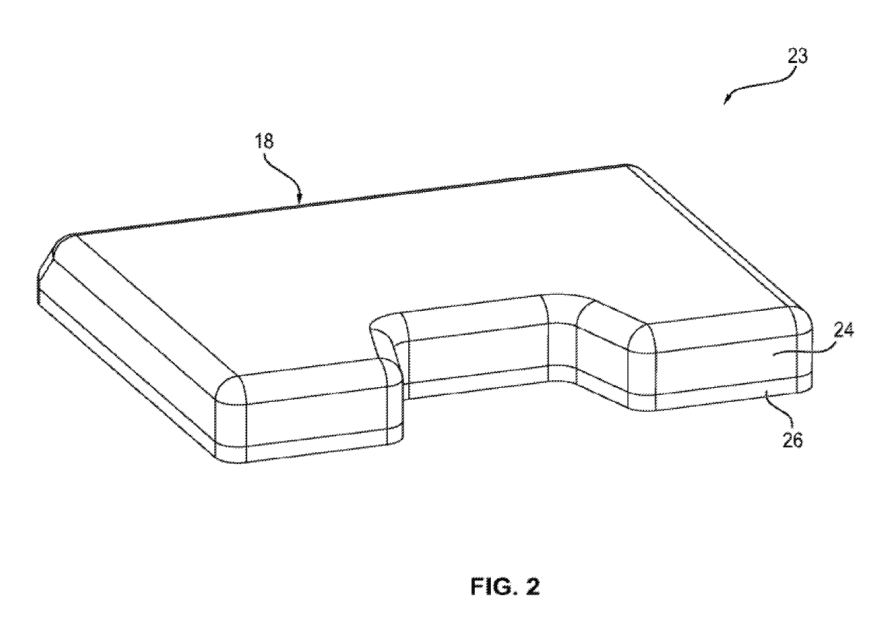

FIG. 2 shows a perspective representation of the pelvis plate 18 according to a first embodiment. The pelvis plate 18 comprises a cushion unit 24 and a support plate 26 connected to it.

The pelvis plate 18 arrangement is a particular example and deployment of a warmed bearing area segment. The same structures and methods can be applied to provide heated bearing area segments in other shapes and positions, to entire beds or sections of beds, and the like. Teachings regarding the pelvis plate example should be understood as applicable to other bearing area segments, to bed and surgical tables generally, and resilient support arrangements generally.

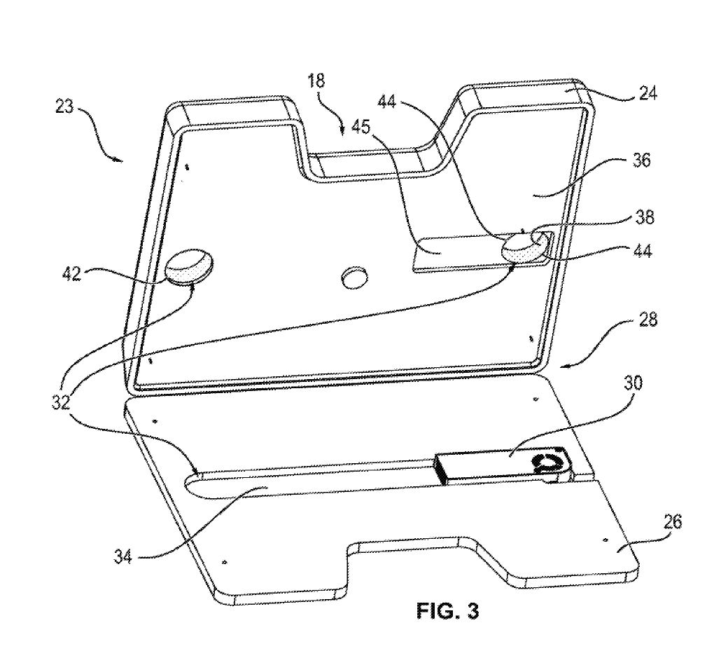

FIG. 3 shows a perspective view of the pelvis plate 18, wherein the cushion unit 24 and the support plate 26 are shown not connected for purposes of illustration, so that elements arranged in the pelvis plate 18 are visible. The pelvis plate 18 comprises a combined heat generating and recirculation unit 30 and a flow channel system 32 through which heated air can flow, formed in the support plate 26 and the cushion unit 24. In other embodiments, the heat generator and recirculation unit may be separate elements arranged sequentially, in either order.

A first flow channel of the flow channel system 32 is formed by a cavity 34 in the support plate 26. The support plate 26 in the connected state shown in FIG. 2 makes contact with an internal support plate 36 of the cushion unit 24, visible in FIG. 3. At the side of the internal support plate 36 facing away from the support plate 26 there is arranged an elastically deformable, air-impermeable cushion layer 38 of the cushion unit 24, whose air permeability is substantially less than that of the air-permeable flow layer 46.

The cushion layer 38 and the internal support plate 36 have a feed opening 42 and an exit opening 44, which are each formed as a common through hole in the cushion layer 38 and the internal support plate 36. The internal support plate 36 has a cavity 45, into which protrudes the heat generating and recirculation unit 30 arranged in the cavity 34 of the support plate 26 when the support plate 26 and the internal support plate 36 lie against each other. In other embodiments there may be two or more exit openings and feed openings.

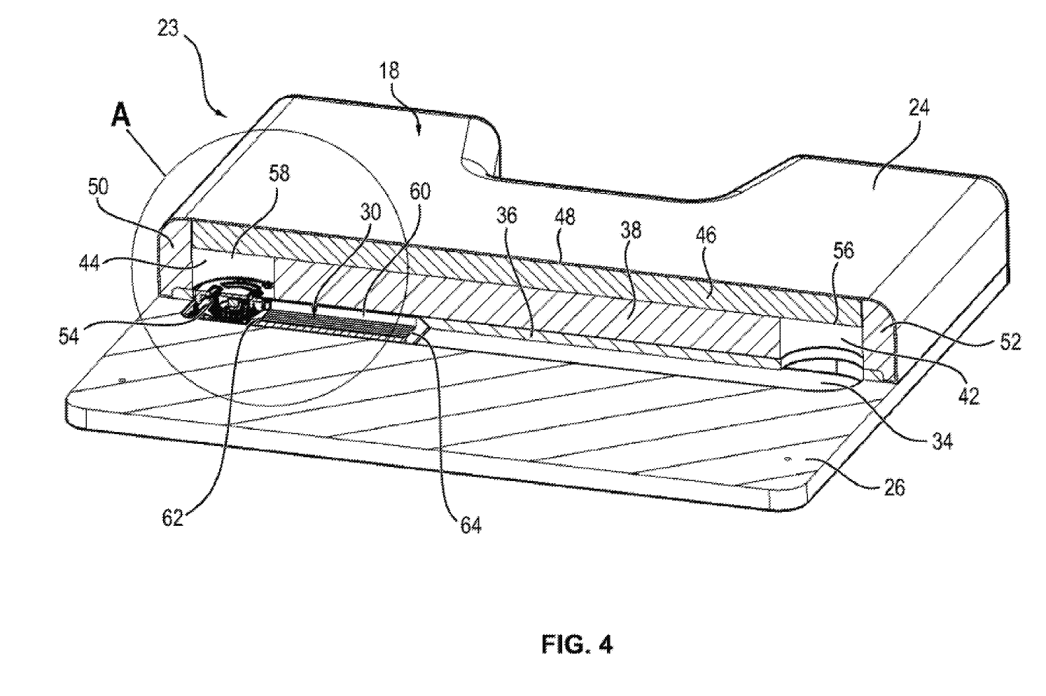

FIG. 4 shows a partly sectioned perspective representation of the pelvis plate 18. The cushion unit 24 comprises the internal support plate 36, the cushion layer 38, the feed opening 42, an air-permeable flow layer 46, an air-impermeable sealing layer 48, a first air-impermeable side cushion 50, a second air-impermeable side cushion 52, and the exit opening 44. The feed opening 42 here forms a second flow channel of the flow channel system 32, the flow layer 46 a third flow channel of the flow channel system 32, and the exit opening 44 a fourth flow channel of the flow system 32.

The flow layer 46 has an entry region 56, which is arranged at a first end of the feed opening 42. The second end opposite the first end of the feed opening 42 borders on the cavity 34 of the support plate 26, in which the heat generating and recirculation unit 30 is arranged. The heat generating and recirculation unit 30 has a recirculation unit 54 and a heating element 60, having an air entry opening 62 and an air exit opening 64. The recirculation unit 54 sucks in air through the exit opening 44 of an exit region 58 of the flow layer 46 and takes it to the heating element 60, which then heats it.

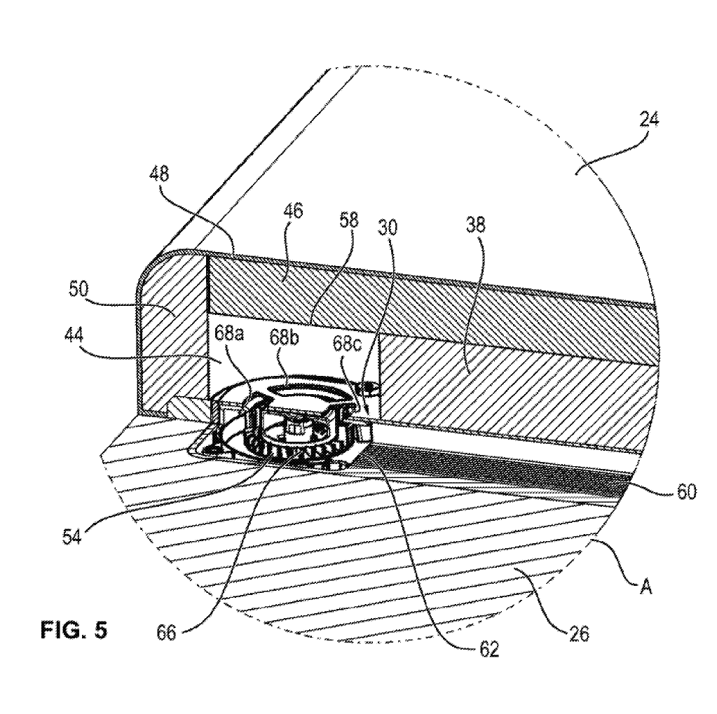

FIG. 5 shows an enlarged representation of a region A indicated in FIG. 4, which comprises the heat generating and recirculation unit 30. The recirculation unit 54 has entry openings 68a, 68b and 68c oriented in the direction of the exit opening 44 and a radial fan 66, which sucks in air centrally from the entry openings 68a, 68b and 68c and conducts it via the air entry opening 62 to the heating element 60. Other types of recirculation units are possible including, without limitation, other types of fans, blowers, and pumps.

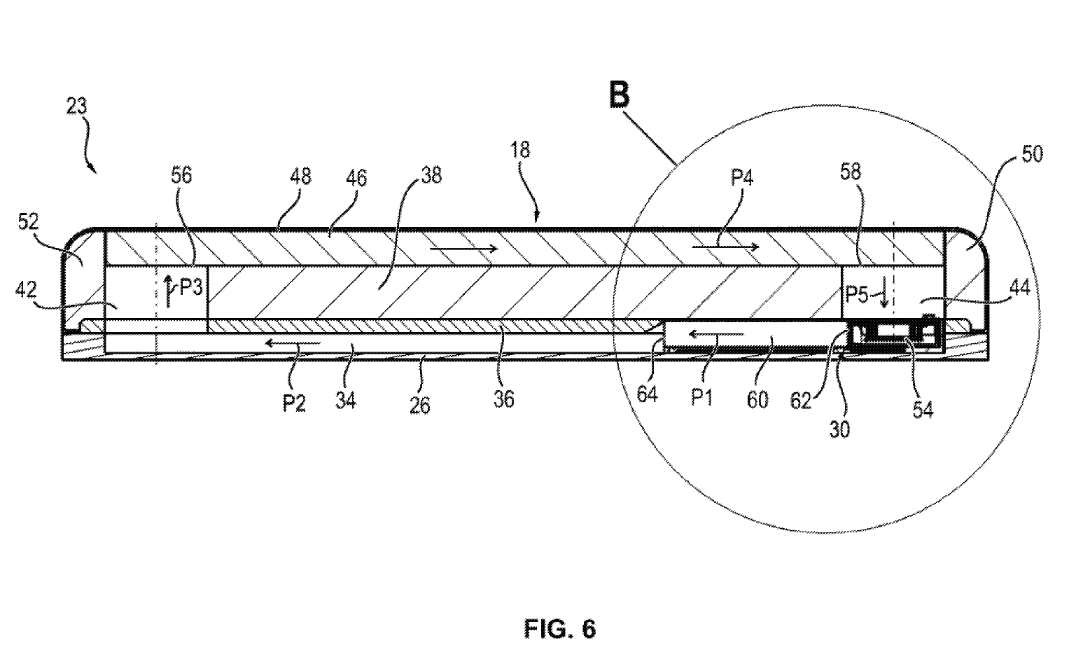

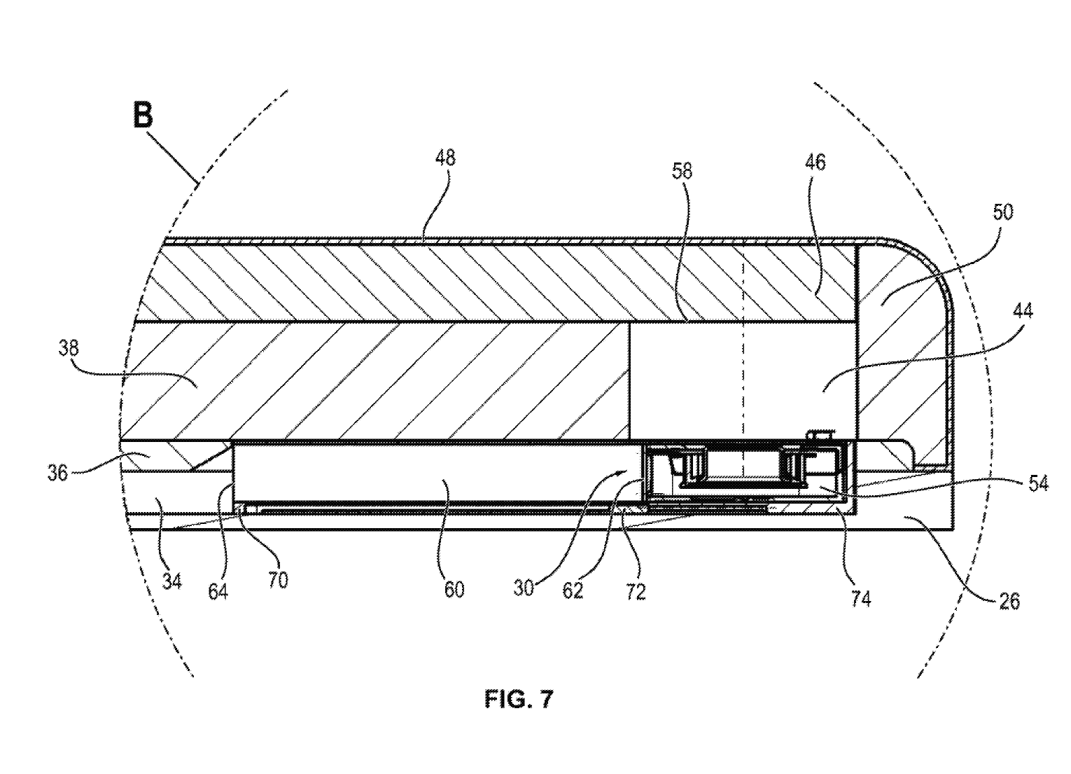

FIG. 6 shows a cross section of the pelvis plate 18 along a vertical sectioning plane transverse to the patient bearing area 10, of which FIG. 7 shows a region indicated as B in FIG. 6 in an enlarged representation. The recirculation unit 54 generates an air flow in the flow channel system 32 of the pelvis plate 18, whose direction in the four flow channels and in the heat generating and recirculation unit 30 is shown each time by arrows P1 to P5, indicated in FIG. 6. In this example the flow layer 46, feed opening 42, exit opening 44, cavity 34, heating element 60, and recirculation unit collectively form a directional path or circuit, with the fluid passing through each of the elements repeatedly and with each trip through the circuit. Person of skill will understand that there is some flexibility in the sequence of the elements. For example, the order of the cavity, heating element, and recirculation unit can be varied.

When the radial fan 66 is activated, the recirculation unit 54 sucks in air from the exit opening 44 and supplies it to the heating element 60 via its air entry opening 62, so that the air flows past a heat exchanger of the heating element 60 in the direction of the arrow P1 and emerges via the air exit opening 64 into the first flow channel formed by the cavity 34. In the first flow channel, the air flows in the direction of the arrow P2 into the feed opening 42, along the arrow P3 in this and across the entry region 56 into the flow layer 46. In the flow layer 46, the air flows in the direction of the arrow P4 and thereby heats the flow layer 46 and the sealing layer 48. After flowing through the flow layer 46, the cooled air flows across the exit region 58 from the flow layer 46 into the exit opening 44 in the direction of arrow P5.

The air is then sucked in again by the recirculation unit 54, so that it has traveled once through the closed flow circuit formed by the flow channels. In other embodiments, the air may also flow in the direction opposite the arrows P1 to P5 if another correspondingly designed recirculation unit is used with reversed direction of flow.

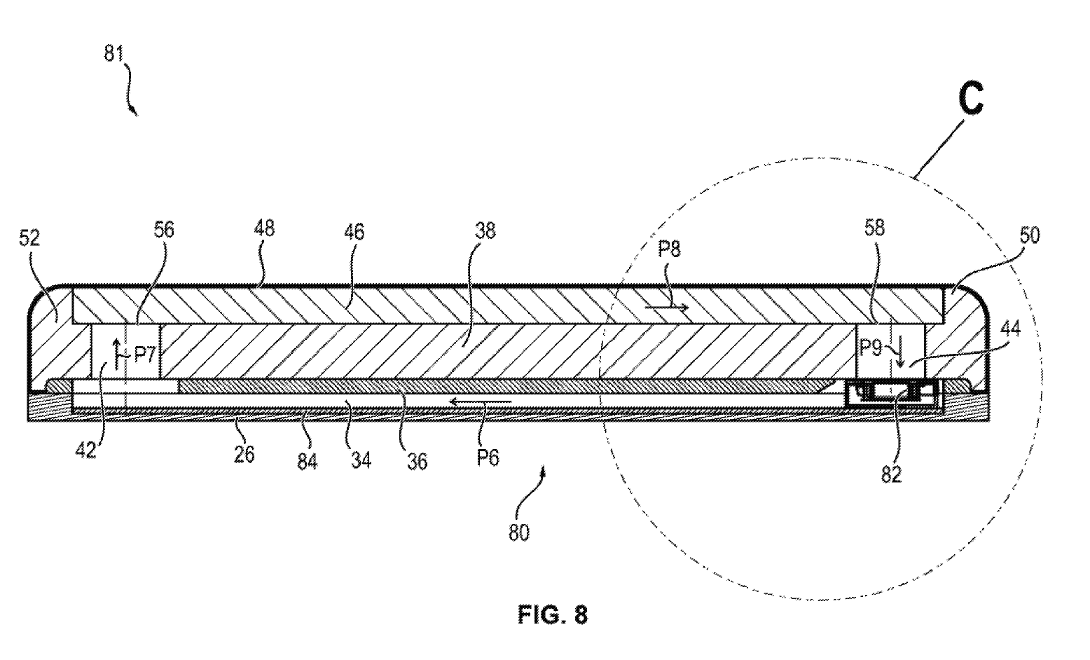

FIG. 8 shows a cross section of a bearing area segment 80 of a device 81 for heating the bearing area segment 80 according to a second embodiment. The bearing area segment 80 has a similar construction to the pelvis plate 18 of the first embodiment. Elements with the same construction or the same function have the same reference numbers. Instead of the heat generating and recirculation element 30 of the pelvis plate 18 of the first embodiment, there are provided in the bearing area segment 80 of the second embodiment a recirculation unit 82 and a surface heating element 84. The surface heating element may be embodied as one or more heated walls along the fluid flow path. For example, as one or more heated walls along the fluid flow path, typically outside of the flow layer 46. In the FIG. 8 example the surface heating element 84 forms a heated wall or floor of the cavity 34 which the fluid flows through on its way back to the flow layer.

The arrows P6 to P9 indicated in FIG. 8 show the direction of the fluid flow (in this example, air flow) when the recirculation unit 82 is activated. The air flow upon flowing through the cavity 34 is heated by the surface heating element 84. The heated air then enters the feed opening 42. The same principles are applicable to fluids other than air, such as liquids.

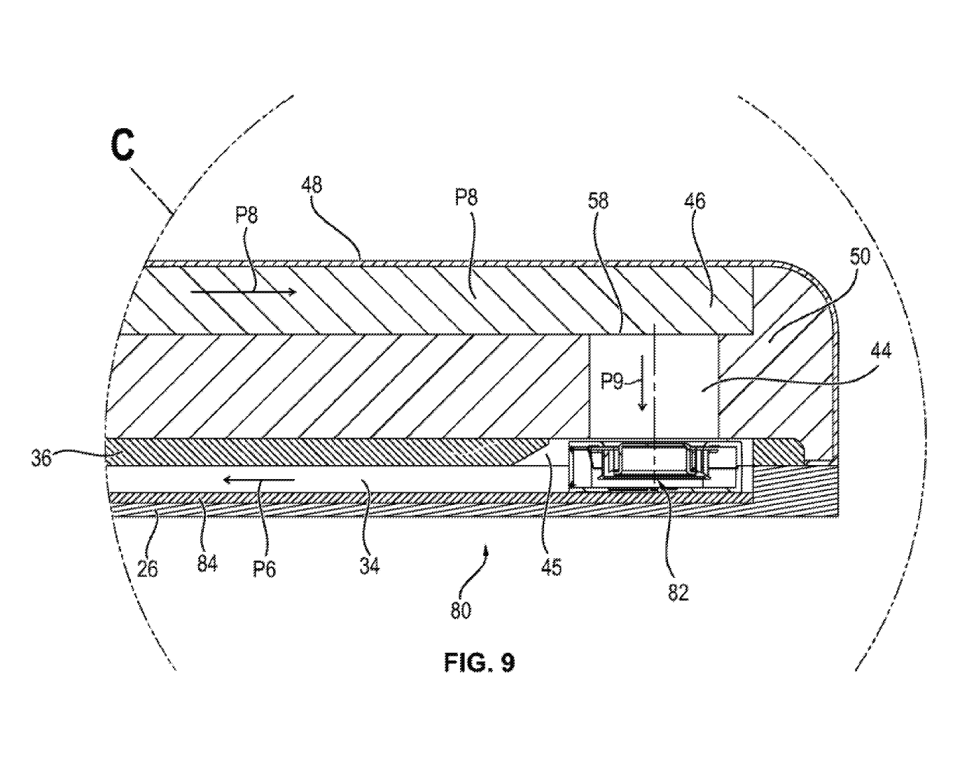

FIG. 9 is an enlarged representation of a region of the bearing area segment 80 designated as C in FIG. 8, where the recirculation unit 82 contacts the surface heating element 84 and the cushion layer 38, so that no seals are required.

FIG. 10 shows a perspective representation of an enlarged partly sectioned cutout of the bearing area segment 80 in which the recirculation unit 82 is arranged. The recirculation unit 82 has an air entry opening 86, through which air is sucked in from the exit opening 44, and an exit opening 88 of the recirculation unit 82, through which the air flow emerges into the cavity 34 and flows in the flow channel 34 past the surface heating element 84, thereby becoming heated.

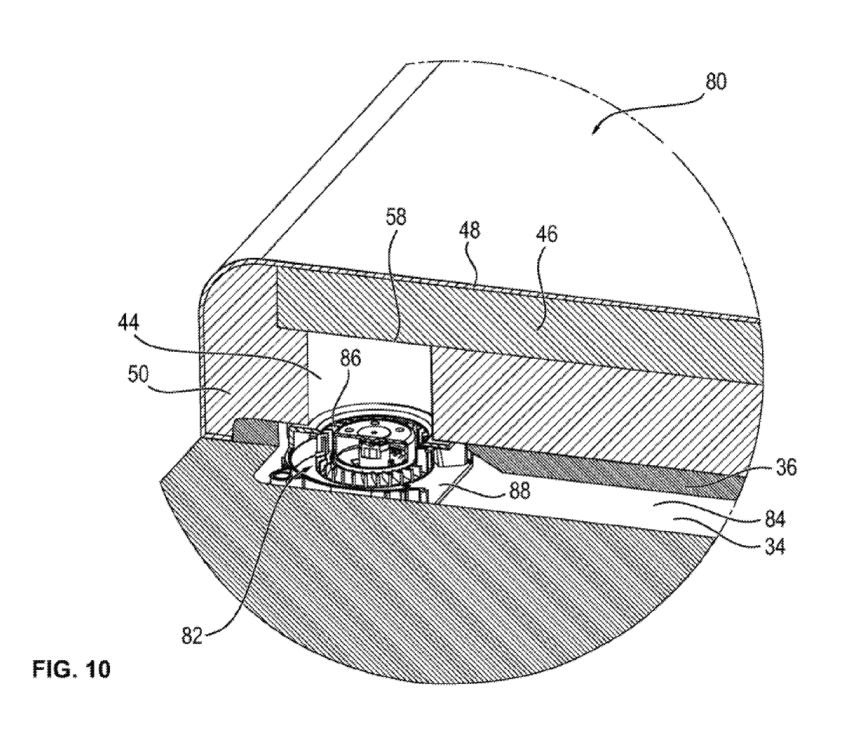

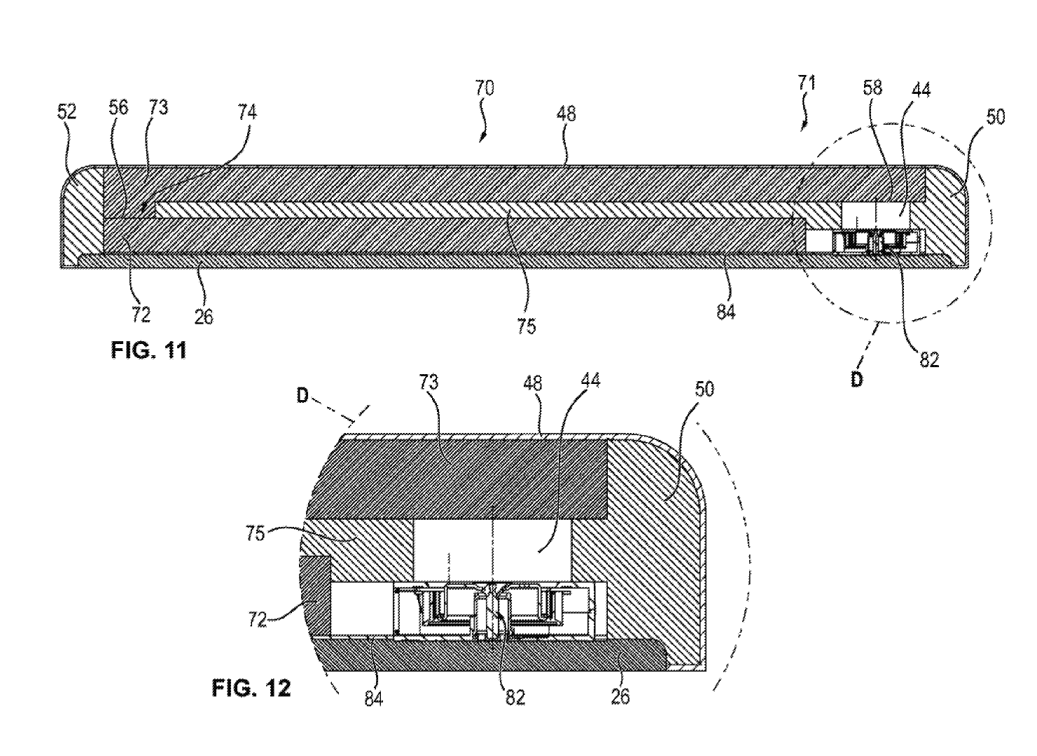

FIG. 11 shows a cross section of a bearing area segment 70 of a device 71 for heating the bearing area segment 70 according to a third embodiment. The third embodiment differs from the second embodiment in that the air flow is not taken in a free flow channel 34 across the surface heating element 84, but instead is taken through an additional air-permeable flow layer 72 arranged above the surface heating element 84 and heated in this. For example, through a second resilient flow layer. For example, open-cell foam. In this example the bearing area segment 70 has a cushion layer 75 between two flow layers 72, 73. In one embodiment a non-porous, fluid-impermeable layer 75 separates two fluid-permeable flow layers 72,73. For example, a generally planar, fluid-tight layer 75 separating two permeable flow layers 72, 73. A separating planar layer 75 having an area equal or nearly equal to (e.g. at least 80% or at least 90%) the area of the heated surface of the segment 70 is one embodiment. In some embodiments an interface region 74 (where the flow layers 72, 73 meet and contact each other) replaces the feed opening 42. In this way, the structural height of the bearing area segment 70, and of the overall system, can be reduced.

FIG. 12 is an enlarged representation of the region D of the bearing area segment 70 indicated in FIG. 11. In FIG. 13, a perspective representation of a partly sectioned enlarged cutout of the bearing area segment 70 is shown. In the embodiments of FIGS. 12 and 13 the air flow is conducted above the internal support plate 36 substantially in materials with cushioning properties.

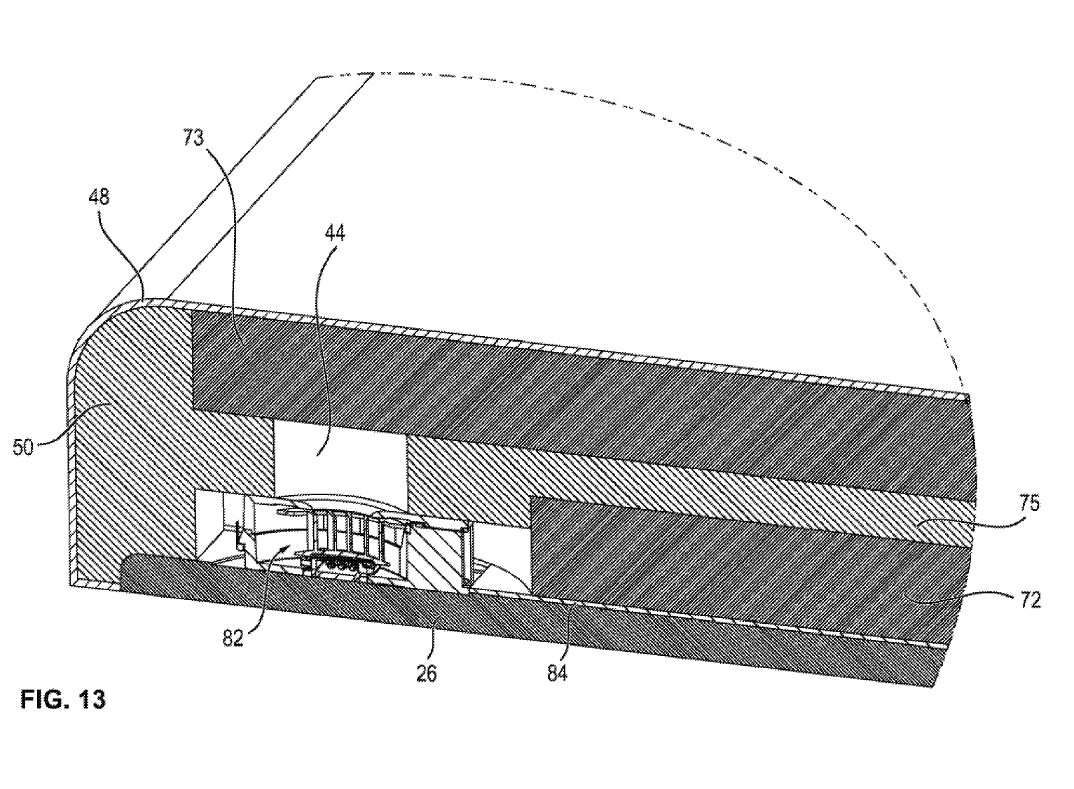

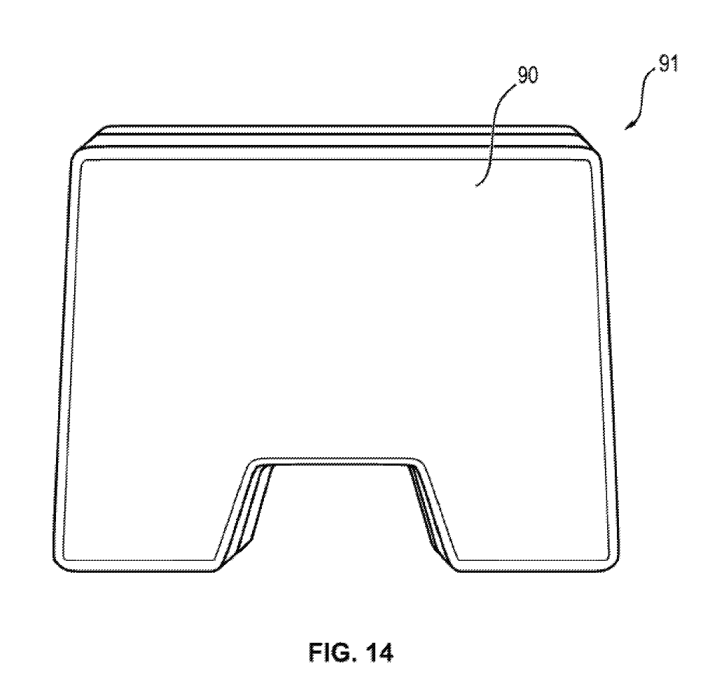

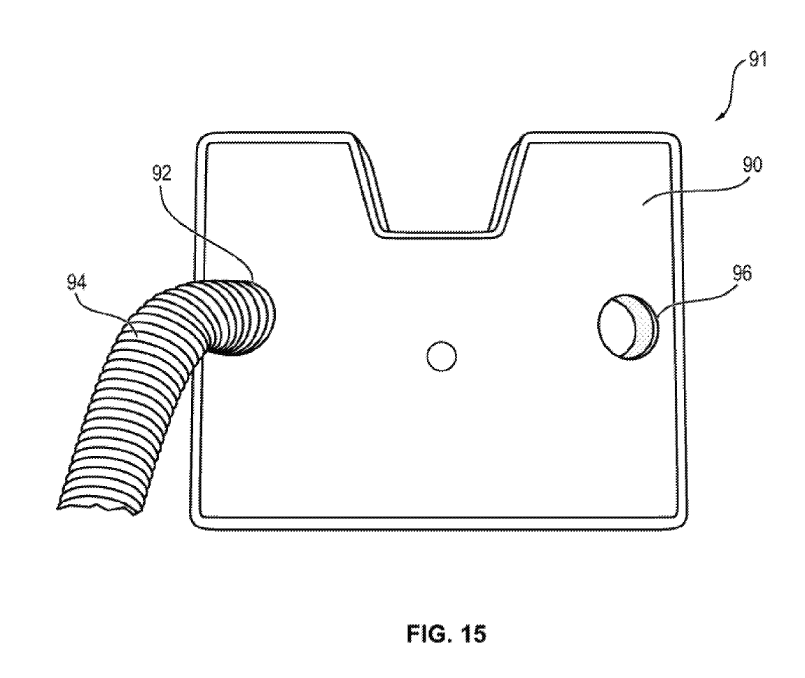

FIG. 14 shows a top view of a bearing area segment 90 of a device 91 according to a fourth embodiment, whose bottom is shown in FIG. 15. The bearing area segment 90 has a first port 92 on its inner side, to which a first hose 94 is connected, and a second port 96, to which a second hose not represented in FIG. 15 is connected.

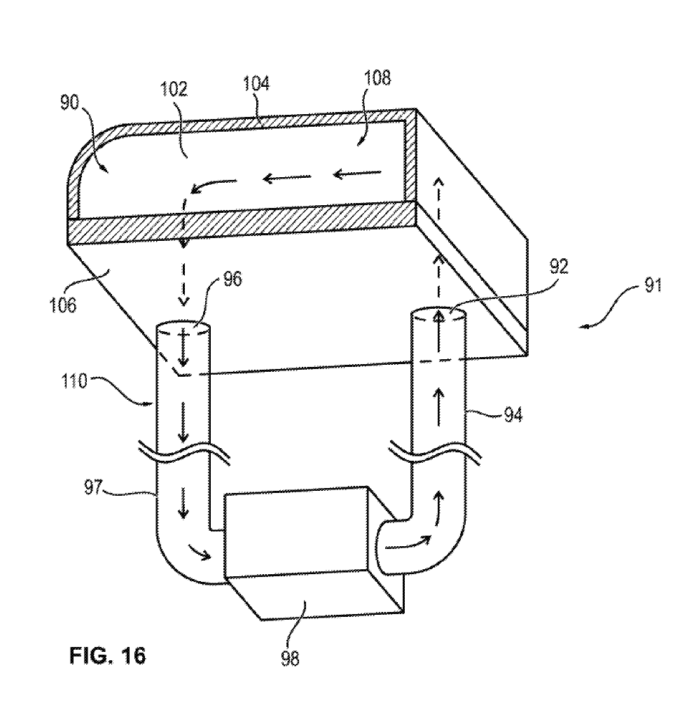

FIG. 16 shows a schematic perspective representation of a cross section of the bearing area segment 90 and an external heat generating and recirculation unit 98. The heat generating and recirculation unit 98 is connected via the first hose 94 to the first port 92 of the bearing area segment 90 and via the second hose 97 to the second port 96 of the bearing area segment 90.

The bearing area segment 90 has an air-permeable flow layer 102, an air-impermeable sealing layer 104 and a support plate 106. The flow layer 102 and the sealing layer 104 form an elastically deformable cushion 108. Moreover, the flow layer 102, the first port 92, the first hose 94, the second port 96, the second hose 97 and the heat [generating] and recirculation unit 98 form a flow channel system 110 of the device 91, through which air flows in a closed circuit.

The flow layer 102 receives a flow of fluid (e.g. air) heated by the heat generating and recirculation unit 98 in the direction of the indicated chain of arrows and is thereby heated. The air then emerges from the second port 96 of the bearing area segment 90 and is taken by the second hose 97 to the heat generating and recirculation unit 98. The heat generating and recirculation unit 98 heats and cleans the air which has become cooled down by heating the flow layer 102 and takes it by the first hose 94 across the first port 92 to the bearing area segment 90 once more. Otherwise, the construction and function of the device 91 may correspond to the device 23.

In a device according to a fifth embodiment, not represented, the air flows in an open circuit. The heat generating and recirculation unit 98 sucks air in from the surroundings, heats and cleans it and takes it through the first hose 94 via the first port 92 to the bearing area segment 90. After flowing through the flow layer 102, the air emerges from the second port 96 into the surroundings. The further construction and function of the device according to the fifth embodiment correspond to those of the device 91.

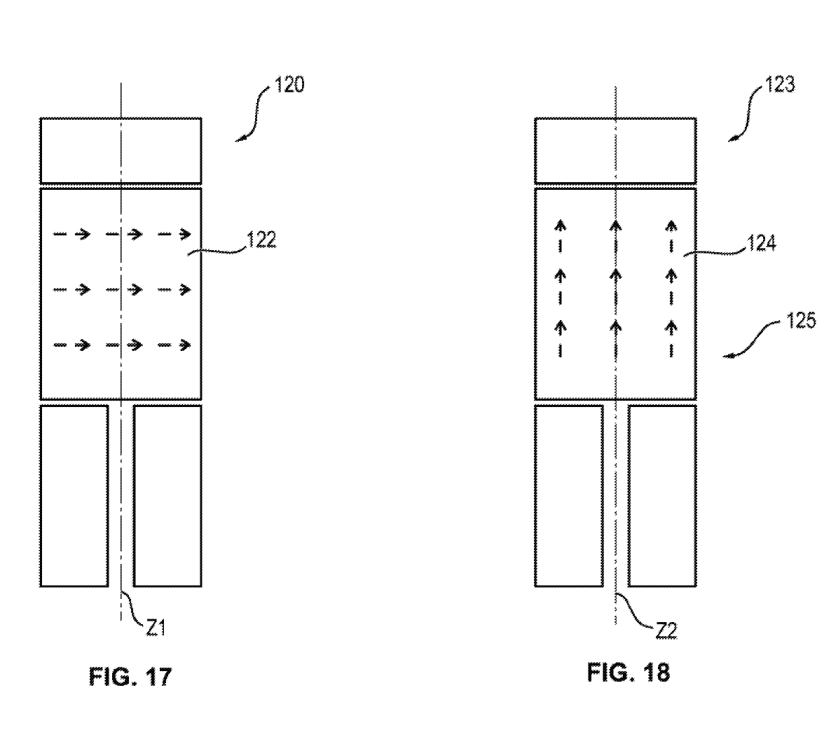

FIG. 17 shows a top view of a schematically represented patient bearing area 120, having a bearing area segment 122 receiving a flow transversely to the longitudinal axis Z1 of the patient bearing area 120, which in the present embodiment serves as a pelvis plate. Otherwise, the construction and function of the bearing area segment 122 correspond to the pelvis plate 18.

FIG. 18 shows a top view of a schematically represented patient bearing area 123, having a bearing area segment 124 receiving a flow of air in the direction of the longitudinal axis Z2 of the patient bearing area 123 of a device 125 for heating the bearing area segment 124. The entry region of the flow layer and the exit region of the flow layer in the case of the bearing area segment 124 of the patient bearing area 123, unlike the bearing area segments 18, 80 and 90 of the embodiments one through five, are arranged at a spacing along the longitudinal axis Z2, so that the bearing area segment 124 receives a lengthwise flow of air. Otherwise, the construction and function of the bearing area segment 124 correspond to the bearing area segment 18.

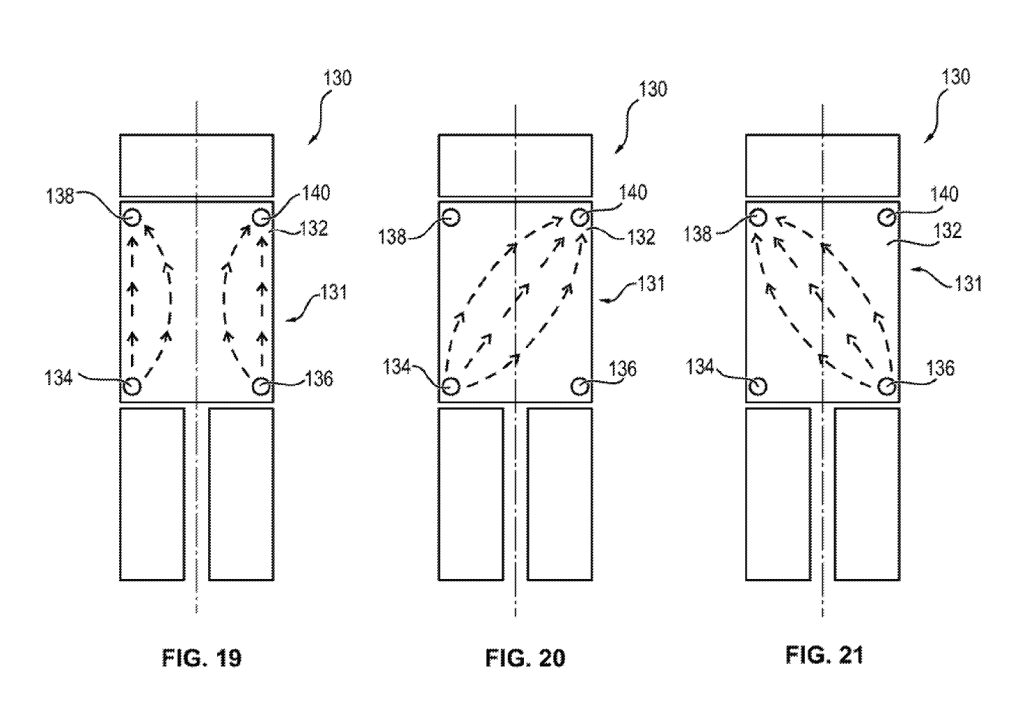

FIGS. 19, 20 and 21 each show a top view of a schematically represented patient bearing area 130, having a device 131 for heating a bearing area segment 132 according to a sixth embodiment. FIGS. 16 to 18 show different configurations for the flow of air through the bearing area segment 132. The bearing area segment 132 has a flow layer with a first entry region 134 and a second entry region 136, each of which has an inlet for introducing heated air into the bearing area segment 132, a first exit region 138 and a second exit region 140, each of which has an outlet for taking away the cooled air. The further construction of the device 131 corresponds to that of the third embodiment. Valve and/or gate arrangements may be provided in order to specifically supply heated air to the entry region and specifically allow air to escape from the exit region.

In the configuration shown in FIG. 19, the flow layer is supplied with heated air through the entry regions 134 and 136 at the same time. The heated air flows through the flow layer along the bearing area segment 132, heats the flow layer and exits from the exit regions 138 and 140 from the flow layer.

The flow layer shown in FIG. 20 is supplied with heated air via the first entry region 134. The heated air flows diagonally through the flow layer of the bearing area segment 132 and exits from the flow layer through the second exit region 140.

In the configuration shown in FIG. 21, the flow layer is supplied with heated air via the second entry region 136. The heated air flows diagonally through the flow layer of the bearing area segment 132 in the direction of the first exit region 138 and exits from the flow layer through this. Especially in the configurations shown in FIG. 20 and FIG. 21, heated air can be supplied to the entry regions with the aid of a valve system, actuated by a control unit, and the flow through the bearing area segment 132 shown in the respective figure can be achieved. For example, the flow through the bearing area segment shown in FIGS. 20 and 21 can be generated alternately in a sequence with the aid of the control unit.

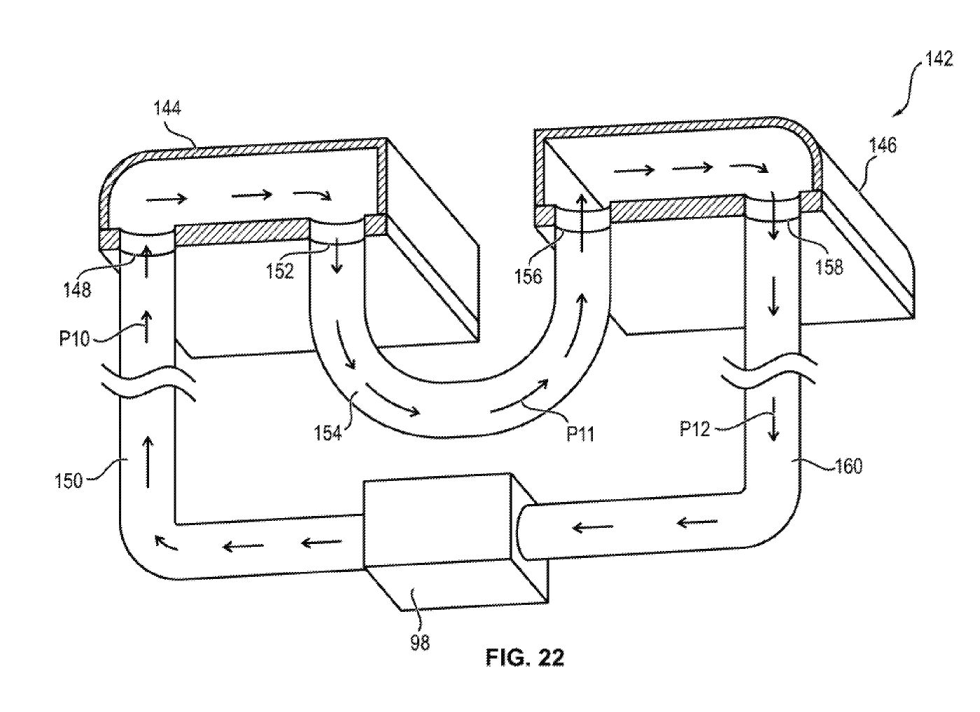

FIG. 22 shows a schematic perspective view of a device 142 for the heating of two bearing area segments 144, 146 receiving an air flow in sequence according to a seventh embodiment. The first bearing area segment 144 has a first port 148, to which a first end of an air feed 150 is connected, and a second port 152, to which a first end of an air connection 154 is connected.

Moreover, the second bearing area segment 146 has a first port 156, to which the second end of the air connection 154 is connected, and a second port 158, to which an air return 160 is connected. The other end of the air return 160 and the other end of the air feed 150 are connected to the heat generating and recirculation unit 98. The further construction of the bearing area segments 144 and 146 corresponds to that of the bearing area segment 90.

The heat generating and recirculation unit 98 creates an air flow, which flows through the air feed 150 in the direction of the arrow P10 and enters via the first port 148 into the flow layer of the first bearing area segment 144. After flowing through the flow layer of the first bearing area segment 144, the air emerges from the second port 152 from the first bearing area segment 144, flows through the air connection 154 in the direction of arrow P7 to the second port 156 and arrives through the flow layer of the second bearing area segment 146. After flowing through the flow layer of the second bearing area segment 146, the cooled air emerges via the second port 158 from the second bearing area segment 146 and is taken via the air return 160 in the direction of arrow P8 to the heat generating and recirculation unit 98, which again heats the air.

Instead of the pelvis plates 18 and 122 described in connection with the figures, the devices 23, 71, 81, 91, 125, 131 and 142 can be used in connection with any other bearing area segments for heating. In all embodiments air or fluids other than air can be used. For example, other gasses, mixtures of gasses, or liquids.

Embodiments include a device 23 for heating a patient bearing area 10 of an operating table, with a heating element 60 for heating a fluid that transfers heat, an elastically deformable flow layer 46 of the patient bearing area 10 through which the heated fluid can flow, and a fluid-tight sealing layer 48 of the patient bearing area 10 arranged between a surface of the patient bearing area 10 provided for contact with the patient and said flow layer 46. Some embodiments are characterized in that the flow layer 46 has at least one entry region 56 for introducing the fluid heated by the heating element 60 into the flow layer 46, and the flow layer 46 has at least one exit region 58 for the emergence of the fluid from the flow layer 46. In some embodiments a flow generator 54 is provided, by means of which a fluid flow can be created for the introducing of the fluid into the entry region 56 of the flow layer 46, for the flow through the flow layer 46 and for the emergence from the exit region 58 of the flow layer 46.

In some embodiments the patient bearing area 10 has at least one bearing area segment 18 with a cushion 24, the surface of the patient bearing area 10 is a surface of the cushion 24 of the bearing area segment 18, and the flow layer 46 and the sealing layer 48 arranged in the cushion 24 of the bearing area segment 18. The flow layer 46 may be formed by an open-pore foam material. A surface of the patient bearing area 10 may be provided for contact with the patient, formed as a surface of the sealing layer 48. In some embodiments a heat storage layer adjacent to the flow layer 46 is provided for the storage of the heat of the fluid. In alternative embodiments at least one entry region 56 and the at least one exit region 58 are arranged outside a region of the patient bearing area 10 intended for the radioscopy of the patient with imaging methods. Some embodiments are characterized in that the heating element 60 is arranged in the patient bearing area 10. In some embodiments the heating element is an X-ray transparent surface heating element 84, and when the flow generator 54 is activated the fluid flows past the surface heating element 84.

In some embodiments the entry region 56 is arranged at a first end of the bearing area segment 124 and the exit region 58 is arranged at a second end of the bearing area segment 124 opposite the first end in the longitudinal direction (Z2) of the patient bearing area 123. The flow layer 102 of the bearing area segment 132 may have another entry region 136 for the introducing of fluid heated by the heating element 98 into the flow layer 102, and/or the flow layer 102 of the bearing area segment 132 may have another exit region 140 for the emergence of the fluid from the flow layer 102. In some embodiments a control unit and a valve system are provided for controlling the fluid flow through the flow layer 102, and the valve system can be controlled by the control unit such that the fluid is introduced only in an entry region 134, 136 into the flow layer 102. A cleaning unit may be provided for cleaning of contaminated fluid.

In some embodiments the bearing area segment is a first bearing area segment 144, the first bearing area segment 144 has a first port 148 for the introducing of the fluid into the first bearing area segment 144 and a second port 152 for the emergence of the fluid from the first bearing area segment 144, the patient bearing area has at least one second bearing area segment 146 with a first port 156 for the introducing of the fluid into the second bearing area segment 146 and one second port 158 for the emergence of the fluid from the second bearing area segment 146, the second bearing area segment 146 has an elastically deformable flow layer through which heated fluid can flow and a fluid-impermeable sealing layer arranged between a surface of the second bearing area segment 146 designed for patient contact and the flow layer of the second bearing area segment 146, the flow layer of the second bearing area segment 146 has at least one entry region for the introducing of the fluid heated by the heating element 98 into the flow layer of the second bearing area segment 146 and at least one exit region for the emergence of the fluid from the flow layer of the second bearing area segment 146, and the second port 152 of the first bearing area segment 144 is connected to the first port 156 of the second bearing area segment 146 such that the fluid, when the flow generator 98 is activated, is introduced by the first port 148 of the first bearing area segment 144 into the first bearing area segment 144, flows from the first bearing area segment 144 into the second bearing area segment 146, is introduced into the entry region of the flow layer of the second bearing area segment 146, emerges from the exit region of the flow layer of the second bearing area segment 146 and emerges from the second port 158 of the second bearing area segment 146.

Some embodiments are characterized by a closed fluid circulation, in which the flow layer 46, the heating element 60 and the flow generator 54 are arranged. In other embodiments air is provided as the fluid, and air can be sucked in from the surroundings by means of the flow generator 98, and the air sucked in from the surroundings when the flow generator 98 is activated is introduced into the entry region of the flow layer 102 of the bearing area segment 90.

The foregoing description of embodiments of the present disclosure is presented for the purpose of illustration and description only, and is not to be construed as limiting the scope of the invention in any way. It is intended that the specification and the disclosed examples be considered as exemplary only, and that the examples not be limiting on the disclosure.

1: A patient bearing area for use as part of an operating table for holding and heating a patient during a medical procedure, the patient bearing area comprising:
a plurality of bearing area segments for collectively supporting the patient;
wherein at least some of the bearing areas segments each comprise a top surface oriented for supporting the patient;
wherein said bearing area segments comprise a heated segment for warming the patient;
said heated segment comprising:
a circulating fluid;
said top surface, the top surface being fluid-tight, and being oriented for supporting the patient;
a flow layer below the top surface, the flow layer comprising open-cell foam, and being elastically deformable;
a dividing member below the flow layer, the dividing layer being made of fluid impermeable material, and having a feed opening and an exit opening there through;
a flow channel below the dividing member;
a heat generating unit for heating the circulating fluid, the heat generating unit being below the dividing member; and
a recirculating unit located below the dividing member, and oriented for impelling the circulating fluid through a flow circuit in a downstream direction;
wherein the flow circuit is a closed path within the heated segment for the circulating fluid, the flow circuit being collectively formed by at least the following elements: the flow layer, the exit opening of the dividing member, the flow channel, and the feed opening of the dividing member;
wherein the recirculating unit and the heat generating unit are each positioned either in or adjacent to the flow channel for, respectively, impelling and heating the circulating fluid when the circulating fluid is not within the flow layer; and
the flow circuit being arranged so that during operation heated circulating fluid enters the flow layer and flows there through for providing heat to the top surface, and so that circulating fluid thereafter leaves the flow layer and returns to the heat generating unit.

2: The patient bearing area of claim 1:
wherein the dividing member is a rigid, planar, support plate having first and second edges at opposite ends thereof;
wherein the support plate comprises a feed opening there through for circulating fluid entering the flow layer, and an exit opening there through for fluid leaving the flow layer;
wherein at least part of the feed opening is within three inches of the first edge of the support plate, at least part of the exit opening is within three inches of the second edge of the support plate, and the feed opening and exit openings are spaced at least five inches apart from each other.

3: The patient bearing area of claim 1:
wherein the heated segment comprises a bottom plate positioned below both the flow layer and the dividing member, with the dividing member located between the flow layer and the bottom plate;
wherein at least part of the flow channel is in the form of a concave cavity in the bottom plate.

4: The patient bearing area of claim 1:
wherein the heated segment comprises a bottom plate positioned below both the flow layer and the dividing member, with the dividing member located between the flow layer and the bottom plate; and
wherein the heat generating unit and the recirculating unit are both located between the bottom plate and the dividing layer.

5: The patient bearing area of claim 1:
wherein the flow channel is a channel fluidically connecting the exit opening to the feed opening, and wherein the recirculating unit is located in the flow channel and oriented for impelling circulating fluid downstream towards the feed opening.

6: The patient bearing area of claim 1:
wherein the patient bearing area includes two leg plates shaped for holding respective legs of said patient when present; and
wherein the leg plates are each independently movable.

7: The patient bearing area of claim 1:
wherein said bearing area segments further comprise one or more non-heated segments; and
wherein at least some of the bearing area segments of the operating table are movable with respect to other bearing area segments for providing different support configurations.

8: The patient bearing area of claim 1: wherein at least the circulating fluid and the flow layer of the heated segment are X-ray transparent.

9: The patient bearing area of claim 1:
wherein the heat generating unit comprises an X-ray transparent surface heating element which is positioned along the flow circuit for heating circulating fluid therein.

10: The patient bearing area of claim 1:
wherein the feed opening and the exit opening are positioned outside an area of the heated segment which is within a patient imaging area.

11: The patient bearing area of claim 1, wherein either (i) the circulating fluid is a gas, and the recirculating unit comprises a fan, or (ii) the circulating fluid is a liquid, and the recirculating unit comprises a pump.

12: The patient bearing area of claim 1, wherein the heat generating unit and the recirculating unit are provided in a single combined unit, the combined unit having a heating passage for flow of circulating fluid there through, the combined unit being configured to both heat and compel circulating fluid passing through the heating passage.

13: The patient bearing area of claim 1, further comprising:
one or more valves positioned to prevent circulating liquid from at least one of (i) leaving the flow layer via the feed opening or (ii) entering the flow layer via the exit opening.

14: The patient bearing area of claim 1:
wherein the heated segment is generally planar, having a maximum width at least four times greater than a maximum thickness.

15: A method of heating a patient in need thereof, the method comprising:
positioning the patient on the patient bearing area of claim 1;
heating circulating fluid in the flow circuit of the heated segment using the heat generating unit;
impelling the circulating fluid in a downstream direction with regard to the flow circuit using the recirculating unit;
wherein the circulating fluid impelled by the recirculating unit proceeds through the flow channel, then through the feed opening, then through the open-cell foam of the flow layer, then out of the flow layer via the exit opening, and then returns to the recirculating unit; and
wherein heat is transferred from circulating fluid passing through the flow layer to the patient on the heated operating table by passing through the top surface of the heated segment.

16: A heated segment for use with a table for holding a patient thereon, the heated segment comprising:
a circulating fluid;
a top surface, the top surface being fluid-tight, and being oriented for supporting the patient;
a flow layer below the top surface, the flow layer comprising elastically deformable material;
a dividing member below the flow layer, the dividing layer comprising fluid impermeable material, and having a feed opening and an exit opening there through;
a flow space below the dividing member;
a heat generating unit for heating the circulating fluid, the heat generating unit being below the dividing member; and
a recirculating unit located below the dividing member, and oriented for impelling the circulating fluid through a flow circuit in a downstream direction;
wherein the flow circuit is a closed path within the heated segment for the circulating fluid, the flow circuit being collectively formed by at least the following elements: the flow layer, the exit opening of the dividing member, the flow space, and the feed opening of the dividing member; and
wherein the flow circuit is arranged so that, during operation, heated circulating fluid enters the flow layer and flows there through for providing heat to the top surface, and so that circulating fluid thereafter leaves the flow layer and returns to the heat generating unit.

17: The heated segment of claim 16, wherein the flow layer comprises open-cell foam.

18: The heated segment of claim 16:
wherein the heated segment further comprises a bottom plate positioned below both the flow layer and the dividing member, with the dividing member located between the flow layer and the bottom plate; and
wherein the flow space comprises elastically deformable material, the elastically deformable material in the flow space being located between the bottom plate and the dividing member and being part of the flow circuit.

19: The heated segment of claim 16, wherein the flow space comprises a flow channel fluidically connecting the exit opening of the dividing member to the feed opening of the dividing member.

20: The heated segment of claim 16, wherein at least the circulating fluid and the flow layer of the heated segment are X-ray transparent.
